(19) United States
(12) Patent Application Publication
(10) Pub. No.: US 2024/0316140 A1
(43) Pub. Date: Sep. 26, 2024

(54) COMPOSITION FOR LIVER PROTECTION COMPRISING MIXTURE OF PORCINE PLACENTA ENZYMATIC HYDROLYSATE AND ACID HYDROLYSATE

(71) Applicant: UBIO INC., Seoul (KR)

(72) Inventors: Keun Nam KIM, Seoul (KR); Gun Won BAE, Uiwang-si (KR); Jee Sun HWANG, Anseong-si (KR); Sun Myung YOON, Seoul (KR)

(73) Assignee: UBIO INC., Seoul (KR)

(21) Appl. No.: 18/575,048

(22) PCT Filed: Jun. 30, 2021
(86) PCT No.: PCT/KR2021/008308

§ 371 Date: Dec. 28, 2023

(51) Int. Cl. A61K 38/01; A61K 35/50; A61P 1/16

(52) U.S. Cl. CPC A61K 38/012; A61K 35/50; A61P 1/16

(57) ABSTRACT

The present invention relates to: a composition for liver protection, comprising a mixture of a porcine placenta enzymatic hydrolysate and a porcine placenta acid hydrolysate; and a composition for the prevention, amelioration or treatment of liver damage caused by alcohol, drug addiction or hangover. The composition of the present invention has a remarkable effect on liver protection and, in particular, has a remarkable effect on the prevention, amelioration or treatment of liver damage caused by alcohol, and thus can be effectively used in the pharmaceutical field and the food field.

### TECHNICAL FIELD

The present invention relates to a composition for liver protection comprising a mixture of a porcine placenta enzymatic hydrolysate and a porcine placenta acid hydrolysate.

### BACKGROUND ART

The liver is the most active organ in-vivo metabolism among human organs, and acute or chronic disorders may occur due to various causes such as excessive intake of food including fatty ingredients or alcohol, infection of viruses, harmful substances such as various drugs, and malnutrition, thereby resulting in fatty liver, hepatitis, jaundice, cirrhosis, liver cancer, etc. In particular, excessive fat intake or excessive alcohol intake through food causes fatty liver in which lipids accumulate in liver tissue, and in this case, aspartate transaminase (AST), alanine transaminase (ALT), lactate dehydrogenase (LDH), and the like in serum increase.

Meanwhile, a placenta is composed of a blood chorionic membrane and supplies the necessary oxygen and nutrients to the fetus while maintaining contact between the fetus and the mother tissue. In addition, the placenta plays an important role in removing waste produced by the fetus. The placenta contains various nutrients and hormones necessary for the growth of the fetus, and a porcine placenta is widely used for adults, especially for menopausal symptom alleviation and beauty purposes. The placenta includes nucleic acid ingredients such as essential amino acids, melatonin, RNA, and DNA, and growth factors and cytokines such as SOD (Super Oxide Dismutase), which is an antioxidant enzyme, hyaluronic acid, antioxidant, cytokine, placenta peptide, insulin-like growth promotion factor, epidermal growth promotion factor (EGF), and senescent cell activation factor (SCAF), so that it is known to be useful for fatigue recovery and immunity enhancement. Moreover, the porcine placenta has a high homology to the protein structure of the human placenta among the placentas of mammals, and the porcine placenta has been reported as the source of bioactive cytokines that controls cell differentiation and fetal development as an important ingredient of proteins, various nutrients, DNA, and RNA. Due to these characteristics, the porcine placenta has been applied to food and medicine. However, research on the placenta composition that can effectively prevent or alleviate liver damage caused by alcohol, drug addiction, and hangover is still insufficient.

### DISCLOSURE

### Technical Problem

An object of the present invention is to provide a health functional food composition for liver protection comprising a mixture of a porcine placenta enzymatic hydrolysate and a porcine placenta acid hydrolysate as an effective ingredient.

Another object of the present invention is to provide a health functional food composition for preventing or alleviating liver damage caused by alcohol, drug addiction, or hangover, the composition comprising a mixture of a porcine placenta enzyme hydrolysate and a porcine placenta acid hydrolysate as an effective ingredient.

Still another object of the present invention is to provide a pharmaceutical composition for liver protection comprising a mixture of a porcine placenta enzymatic hydrolysate and a porcine placenta acid hydrolysate as an effective ingredient.

Still another object of the present invention is to provide a pharmaceutical composition for preventing or treating liver damage caused by alcohol, drug addiction, or hangover, the pharmaceutical composition comprising a mixture of a porcine placenta enzyme hydrolysate and a porcine placenta acid hydrolysate as an effective ingredient.

### Technical Solution

To achieve the above objects, the present invention provides a health functional food composition for liver protection comprising a mixture of a porcine placenta enzymatic hydrolysate and a porcine placenta acid hydrolysate as an effective ingredient.

Further, the present invention provides a health functional food composition for preventing or alleviating liver damage caused by alcohol, drug addiction, or hangover, the composition comprising a mixture of a porcine placenta enzyme hydrolysate and a porcine placenta acid hydrolysate as an effective ingredient.

In one embodiment of the present invention, the porcine placenta enzymatic hydrolysate may comprise one or more peptides having an amino acid sequence selected from the group consisting of SEQ ID Nos: 1 to 3.

In one embodiment of the present invention, the porcine placenta enzymatic hydrolysate and the porcine placenta acid hydrolysate may be mixed at a weight ratio of 1:0.1 to 10, and preferably, at a weight ratio of 1:0.5 to 5, 1:0.6 to 5, 1:0.7 to 5, 1:0.8 to 5, 1:0.9 to 5, 1:1 to 5, 1:1 to 4, 1:1 to 3, 1:1, 1:2, 1:3, 1:4, or 1:5, but the present invention is not limited thereto.

In one embodiment of the present invention, the porcine placenta enzymatic hydrolysate may be prepared by treating proteinase, and the proteinase may be selected from the group consisting of papain, protease, bromelain, and alkalase, but the present invention is not limited thereto.

In one embodiment of the present invention, the porcine placenta acid hydrolysate may be prepared by treating acid, and the acid may be hydrochloric acid, sulfuric acid, acetic acid, or citric acid, but the present invention is not limited thereto.

In one embodiment of the present invention, the peptide may be included in the porcine placenta enzymatic hydrolysate at a concentration of 0.1 ppm to 100 ppm, and preferably 1 ppm to 25 ppm.

In one embodiment of the present invention, the mixture of the porcine placenta enzymatic hydrolysate and the porcine placenta acid hydrolysate may be included at 1 to 20 wt % with respect to the total weight of the health functional food composition. In addition, the peptide may be included at a concentration of 0.001 ppm to 20 ppm in the entire composition.

In one embodiment of the present invention, the composition may reduce an alkaline phosphatase (ALP) level, an aspartate transaminase (AST) level, or an alanine transaminase (ALT) level in serum.

Further, the present invention provides a pharmaceutical composition for liver protection comprising a mixture of a porcine placenta enzymatic hydrolysate and a porcine placenta acid hydrolysate as an effective ingredient.

Further, the present invention provides a pharmaceutical composition for preventing and treating liver damage caused by alcohol, drug addiction, or hangover, the pharmaceutical composition comprising a mixture of a porcine placenta enzymatic hydrolysate and a porcine placenta acid hydrolysate as an effective ingredient.

In one embodiment of the present invention, the mixture of the porcine placenta enzymatic hydrolysate and the porcine placenta acid hydrolysate may be included at 5 to 30 wt % with respect to the total weight of the pharmaceutical composition. In addition, the peptide may be included at a concentration of 0.005 ppm to 30 ppm in the entire composition.

### Advantageous Effects

The composition according to the present invention has a remarkable effect on liver protection, and in particular, has a remarkable effect on prevention, alleviation, or treatment of liver damage caused by alcohol, and thus can be effectively used in the pharmaceutical field and the food field.

### DESCRIPTION OF DRAWINGS

FIG. 1 is a result showing HPLC patterns of a porcine placenta enzymatic hydrolysate and a porcine placenta acid hydrolysate.

FIG. 2 is an LC/MS chromatogram result of the porcine placenta enzymatic hydrolysate.

FIG. 3 is a chromatogram result of the porcine placenta enzymatic hydrolysate and a peptide (VVVE).

FIG. 4 is a result showing MS/MS patterns of the porcine placenta enzymatic hydrolysate and the peptide (VVVE).

FIG. 5 is a chromatogram result of the porcine placenta enzymatic hydrolysate and a peptide (DGLHLR).

FIG. 6 is a result showing MS/MS patterns of the porcine placenta enzymatic hydrolysate and the peptide (DGLHLR).

FIG. 7 is a chromatogram result of the porcine placenta enzymatic hydrolysate and a peptide (DDFNPSVH).

FIG. 8 is a result showing MS/MS patterns of the porcine placenta enzymatic hydrolysate and the peptide (DDFNPSVH).

FIG. 9 is a result of measuring alcohol dehydrogenase (ADH) activity in liver tissue after administering the porcine placenta enzymatic hydrolysate, a porcine placenta acid hydrolysate, or a mixture of the porcine placenta enzymatic hydrolysate and the porcine placenta acid hydrolysate (Normal: normal control group; Alcohol: negative control group; Silymarin: positive control group; L: low-dose group of porcine placenta mixture; M: medium-dose group of porcine placenta mixture; H: high-dose group of porcine placenta mixture; E-form: administration group of porcine placenta enzymatic hydrolysate; and A-form: administration group of porcine placenta acid hydrolysate).

FIG. 10 is a result of measuring aldehyde dehydrogenase (ALDH) activity in liver tissue after administering the porcine placenta enzymatic hydrolysate, a porcine placenta acid hydrolysate, or a mixture of the porcine placenta enzymatic hydrolysate and the porcine placenta acid hydrolysate (Normal: normal control group; Alcohol: negative control group; Silymarin: positive control group; L: low-dose group of porcine placenta mixture; M: medium-dose group of porcine placenta mixture; H: high-dose group of porcine placenta mixture; E-form: administration group of porcine placenta enzymatic hydrolysate; and A-form: administration group of porcine placenta acid hydrolysate).

### BEST MODE

As a best mode of the present invention, provided is a health functional food composition for liver protection comprising a mixture of a porcine placenta enzymatic hydrolysate and a porcine placenta acid hydrolysate as an effective ingredient.

Further, as a best mode of the present invention, provided is a health functional food composition for preventing or alleviating liver damage caused by alcohol, drug addiction, or hangover, the composition comprising a mixture of a porcine placenta enzyme hydrolysate and a porcine placenta acid hydrolysate as an effective ingredient.

### MODE FOR INVENTION

The present invention provides a health functional food composition comprising a mixture of a porcine placenta enzyme hydrolysate and a porcine placenta acid hydrolysate as an effective ingredient, and a health functional food composition for preventing or alleviating liver damage caused by alcohol, drug addiction, or hangover.

The porcine placenta enzymatic hydrolysate may comprise one or more peptides having an amino acid sequence selected from the group consisting of SEQ ID Nos. 1 to 3.

The peptide may be included in the porcine placenta enzymatic hydrolysate at a concentration of 0.1 ppm to 100 ppm, preferably 1 ppm to 25 ppm.

The term “porcine placenta enzymatic hydrolysate” of the present invention refers to one prepared by treating porcine placenta with proteinase.

The proteinase may be selected from the group consisting of papain, protease, bromelain, and alkalase, but the present invention is not limited thereto.

The term “porcine placenta acid hydrolysate” of the present invention refers to one prepared by treating porcine placenta with acid.

The acid may be hydrochloric acid, sulfuric acid, acetic acid, or citric acid, but the present invention is not limited thereto.

The porcine placenta enzymatic hydrolysate and the porcine placenta acid hydrolysate may be mixed at a weight ratio of 1:0.1 to 10, and may be preferably mixed at a weight ratio of 1:0.5 to 5, 1:0.6 to 5, 1:0.7 to 5, 1:0.8 to 5, 1:0.9 to 5, 1:1 to 5, 1:1 to 4, 1:1 to 3, 1:1, 1:2, 1:3, 1:4, or 1:5, but the present invention is not limited thereto.

The health functional food composition of the present invention may include all foods in a general sense, and may be used with various terms known in the art, such as a functional food and a health functional food.

The term “health functional food” of the present invention refers to foods prepared and processed in the form of tablets, or capsules, powders, granules, liquids, and pills using raw materials or ingredients having functionality useful for the human body. The term “functionality” used herein means controlling nutrients for the structure and function of the human body or obtaining useful effects for health purposes such as physiological effects. The health functional food of the present invention may be prepared by a method commonly used in the art, and may be prepared by adding raw materials and ingredients commonly added in the art during the preparation. In addition, formulations of the above health functional foods may also be prepared without limitation as long as formulations recognized as health functional foods. The health functional food composition of the present invention has the advantages of having no side effects or the like which may occur when taking medicine for a long period of time using the food as a raw material, unlike general medicines, and has excellent portability, and thus may be taken as an auxiliary agent for improving an effect on preventing or alleviating liver damage caused by alcohol, drug addiction, or hangover.

In the health functional food composition according to the present invention, the effective ingredient (the mixture of the porcine placenta enzymatic hydrolysate and the porcine placenta acid hydrolysate) may be included in an amount of 1% to 20% (wt %) with respect to the total weight of the composition, but the present invention is not limited thereto, and a mixed amount of the effective ingredient may be appropriately determined according to each purpose of use such as prevention, health, or treatment.

Formulations of the health functional food may be in the form of powders, granules, pills, tablets, capsules, as well as in the form of any general food or beverage.

The kinds of the food are not particularly limited, and examples of the food to which the substance may be added may include meat, sausage, bread, chocolate, candies, snacks, pizza, ramen, other noodles, gum, dairy products including ice cream, various soups, beverages, tea, drinks, alcohol beverages, vitamin compounds, and the like, and may include all foods in a general sense.

In general, the effective ingredient may be added in an amount of 15 parts by weight or less, and preferably 10 parts by weight or less, with respect to 100 parts by weight of a raw material in the preparation of food or beverage. However, in the case of long-term intake for the purpose of health and hygiene or for the purpose of controlling health, the amount of the effective ingredient may be less than or equal to the above range. In addition, since there is no problem in terms of safety in that fractions from natural substances are used, the present invention may also be used in an amount greater than or equal to the above range.

Among the functional foods according to the present invention, a beverage may contain various flavoring agents or natural carbohydrates as an additional ingredient, like a conventional beverage. The above-described natural carbohydrates may be monosaccharides such as glucose and fructose, disaccharides such as maltose and sucrose, polysaccharides such as dextrin and cyclodextrin, and sugar alcohols such as xylitol, sorbitol, and erythritol. As a sweetening agent, natural sweeteners such as thaumatin and stevia extracts, and synthetic sweeteners such as saccharin and aspartame may be used. A ratio of the natural carbohydrate may be about 0.01 to 0.04 g, and preferably about 0.02 to 0.03 g per 100 mL of the beverage according to the present invention.

In addition, the health functional food composition according to the present invention may contain various nutrients, vitamins, electrolytes, flavoring agents, colorants, pectic acid, and salts thereof, alginic acid and salts thereof, organic acid, protective colloid thickening agents, pH adjusting agents, stabilizing agents, preservatives, glycerin, alcohols, carbonating agents used in carbonated beverages. In addition, the health functional food composition of the present invention may contain flesh for the preparation of natural fruit juice, fruit juice beverage and vegetable beverage. These ingredients may be used independently or in combination. A ratio of the additive is not limited, but the functional food composition of the present invention is generally selected in a range of 0.01 to 0.1 parts by weight based on 100 parts by weight.

Further, the present invention provides a pharmaceutical composition for liver protection, which comprises a mixture of a porcine placenta enzymatic hydrolysate and a porcine placenta acid hydrolysate as an effective ingredient, and a pharmaceutical composition for preventing and alleviating liver damage caused by alcohol, drug addiction, or hangover.

The pharmaceutical composition according to the present invention is not particularly limited in content as long as it includes the effective ingredient, but preferably, the effective ingredient may be included in an amount of 5 to 30 wt % with respect to the total weight of the composition. However, the present invention is not limited thereto. In addition, the peptide may be included at a concentration of 0.005 ppm to 30 ppm in the entire composition. In this case, when the peptide is less than the concentration range, it is difficult to exert a desirable preventive or therapeutic effect, and when the peptide exceeds the concentration range, a change in the expected effect may be insignificant.

The pharmaceutical composition according to the present invention may be formulated and used in the form of an oral dosage form such as powder, a granule, a tablet, a capsule, a suspension, an emulsion, a syrup, and an aerosol, an external application, a suppository, and a sterile injectable solution according to a conventional method, respectively, and may include a suitable carrier, excipient, or diluent commonly used in the preparation of a pharmaceutical composition for formulation.

Examples of the carrier, the excipient, or the diluent may include various compounds or mixtures including lactose, dextrose, sucrose, sorbitol, mannitol, xylitol, erythritol, maltitol, starch, acacia rubber, alginate, gelatin, calcium phosphate, calcium silicide, cellulose, methyl cellulose, microcrystalline cellulose, polyvinyl pyrrolidone, water, methyl hydroxybenzoate, propyl hydroxybenzoate, talc, magnesium stearate, mineral oil, etc.

When the pharmaceutical composition is formulated, it may be prepared by using diluents or excipients such as fillers, heavy agents, binders, wetting agents, disintegrants, surfactants, etc., which are commonly used.

The solid formulation for oral administration may be prepared by mixing the composition with at least one excipient, for example, starch, calcium carbonate, sucrose or lactose, gelatin, or the like. Lubricants such as magnesium stearate and talc may also be used in addition to a simple excipient.

Liquid formulations for oral administration corresponds to suspensions, contents solutions, emulsions, syrups, and the like, and may include various excipients, for example, wetting agents, sweeteners, air fresheners, preservatives, etc., in addition to water and liquid paraffin, which are commonly used simple diluents.

Formulations for parenteral administration include sterilized aqueous solutions, non-aqueous solutions, suspensions, emulsions, lyophilized formulations, and suppositories. As the non-aqueous solution and the suspension, a vegetable oil such as propylene glycol, polyethylene glycol, and olive oil, and an injectable ester such as ethyl oleate may be used. As a base for suppositories, witepsol, macrogol, tween 61, cacao butter, laurin butter, glycerol gelatin, and the like may be used.

A preferable dosage of the pharmaceutical composition according to the present invention may vary depending on a patient's condition, body weight, degree of disease, drug form, administration route, and duration, but may be appropriately selected by those skilled in the art. However, for a preferable effect, the pharmaceutical composition may be administered at 0.0001 to 2,000 mg/kg per day, and preferably at 0.001 to 2,000 mg/kg. The administration may be performed once a day or several times. However, the scope of the present invention is not limited by the above dosage.

According to the present invention, the pharmaceutical composition may be administered to mammals such as rats, mice, livestock, and humans through various routes. The administration may be performed using all routes of administration including, for example, oral, rectal, intravenous, intramuscular, subcutaneous, intrauterine dura, or intracerebroventricular injections.

Hereinafter, the present invention will be described in more detail with reference to examples. These examples are for describing the present invention in more detail, and the scope of the present invention is not limited to these examples.

### Example 1. Preparation of Porcine Placenta Enzymatic Hydrolysate

After thawing a porcine placenta in a thawing machine, the porcine placenta from which foreign substances were removed with top water was put into a meat tenderizer to facilitate blood removal. Thereafter, the porcine placenta was washed several times with 0.9% NaCl, and the porcine placenta from which the blood in the porcine placenta was removed was crushed using a mixer to facilitate hydrolysis. The prepared porcine placenta was added with 3% of proteinase (papain) and hydrolyzed for 20 hours. After the hydrolysis was completed, the proteinase was inactivated by heating, and the porcine placenta hydrolysate was filtered by making contact with a filter aid and adsorbed and purified. 1.2× (w/w) ethanol was then added to a filtrate of the porcine placenta hydrolysate, and after standing for 15 to 20 hours, sugars, proteins, and impurities, which were not sufficiently hydrolyzed, were removed using the filter. After the filtered liquid was concentrated, the resultant was adsorbed and purified using 0.1 to 2% of activated carbon, and the used activated carbon was then removed by filtration using the filter. The purified porcine placenta enzymatic hydrolysate, from which the activated carbon was removed, was antibacterialized using a 0.2 μm filter to sterilize the filtered liquid. Finally, a high-purity placenta extract was obtained.

### Example 2. Preparation of Porcine Placenta Acid Hydrolysate

After thawing the porcine placenta in the thawing machine, 100 kg of the porcine placenta from which foreign substances were removed with top water was added with 70 kg of 35% (v/w) hydrochloric acid, decomposed at 110° C. for 22 hours, and filtered. Thereafter, the filtered decomposition was concentrated, sodium hydroxide was added to the resultant to neutralize the pH 6 to 7, and then adsorbed and purified using activated carbon. The used activated carbon was removed by filtration using a filter. The purified porcine placenta acid hydrolysate, from which the activated carbon was removed, was antibacterialized using a 0.2 μm filter to sterilize the filtered liquid.

### Example 3. Analysis of Porcine Placenta Enzymatic Hydrolysate and Porcine Placenta Acid Hydrolysate

The present inventors performed an experiment to analyze a nitrogen content, an amino acid content, and an HPLC pattern of the prepared porcine placenta enzymatic hydrolysate and porcine placenta acid hydrolysate in order to confirm properties of the porcine placenta enzymatic hydrolysate and the porcine placenta acid hydrolysate.

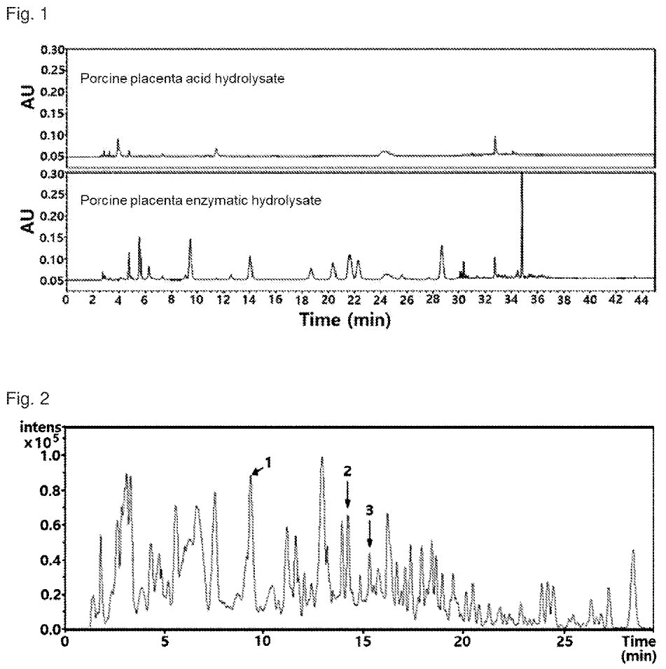

As a result, it was found that the enzymatic hydrolysate had the amino acid content of about 40%, and the porcine placenta acid hydrolysate had the amino acid content of about 80% (Table 1). That is, it was confirmed that the amino acid content of the porcine placenta acid hydrolysate is greater than that of the enzymatic hydrolysate. In addition, as shown in FIG. 1, the HPLC pattern of the porcine placenta enzymatic hydrolysate was different from that of the porcine placenta acid hydrolysate (FIG. 1).

TABLE 1
Analysis of Porcine Placenta Enzymatic Hydrolysate
And Porcine Placenta Acid Hydrolysate

| | Total | Amino | Amino | |
|---|---|---|---|---|
| | nitrogen | acid | acid | Peptide |
| Classification | (mg/mL) | (mg/mL) | % | % |
| Enzymatic hydrolysate | 5.30 | 13.68 | 41.1 | 58.9 |
| Porcine placenta acid | 5.67 | 31.72 | 81.2 | 18.8 |
| hydrolysate | | | | |

$$Amino acid ( \% ) = \frac{\begin{matrix}( Molecular weights of nitrogen and amino acid / \\ Molecular weight of amino acid ) \times \\ Content of amino acid\end{matrix}}{Dilution factor} \times 100 \qquad [ Equation ]$$

$$Peptide ( \% ) = 100 \% - Amino acid \%$$

### Example 4. Identification and Efficacy of Peptide in Porcine Placenta Enzymatic Hydrolysate

### 4.1. Mass Chromatography of Porcine Placenta Enzymatic Hydrolysis

The porcine placenta enzymatic hydrolysate obtained in Example 1 was vortexed by adding 500 μl of methanol (MeOH) to 100 μl of a sample, the sample was centrifuged to transfer 600 μl of a supernatant to a new test tube and was then vacuum-dried. Thereafter, water was added to the dried sample to be 100 μL, the filtered liquid was sequenced using a MicroQ-TOF III mass spectrometer (Bruker Daltonics, 255748 Germany) system and ms/ms ionization analysis.

### <Analysis Condition>

### Mobile Phase:

Mobile Phase A: H₂O/FA=100/0.2 (v/v)

Mobile Phase B: Acetonitrile/FA=100/0.2 (v/v)

TABLE 2
Analysis Condition of Mass Chromatography

| Step (%) | Total Time (min) | Flow Rate (μl/min) | A (%) | B (%) |
|---|---|---|---|---|
| 0 | 0.00 | 200 | 95 | 5 |
| 1 | 5.00 | 200 | 95 | 5 |
| 2 | 28.00 | 200 | 70 | 30 |
| 3 | 33.00 | 200 | 5 | 95 |
| 4 | 40.00 | 200 | 5 | 95 |
| 5 | 41.00 | 200 | 95 | 5 |
| 6 | 46.00 | 200 | 95 | 5 |

As a result of the above analysis, a total of 17 peptides were analyzed, and 5 peptides were identified as a porcine-derived peptide in an UniProt database (Table 3).

TABLE 3
Peptide Sequence of Porcine Placenta Enzymatic Hydrolysate

| No. | m/z | RT (min) | Charge | Sequence | Organism |
|---|---|---|---|---|---|
| 1 | 445.27 | 10.0 | 1 | VVVE | Sus scrofa (Pig) |
| 2 | 571.27 | 11.5 | 1 | QMHR | — |
| 3 | 355.70 | 14.5 | 2 | DGLHLR | Sus scrofa (Pig) |
| 4 | 394.73 | 14.7 | 2 | LDKWNL | Sus scrofa (Pig) |
| 5 | 438.24 | 15.3 | 2 | SLDKRAK | — |
| 6 | 465.71 | 15.4 | 2 | DDFNPSVH | Sus scrofa (Pig) |
| 7 | 490.23 | 15.9 | 1 | GPLCT | Sus scrofa (Pig) |

Thereafter, in the present invention, three porcine-derived peptides were selected as index substances by confirming a peak size and interbatch reproducibility among the identified five porcine-derived peptides (FIGS. 2 and Table 4).

TABLE 4
Peptide Sequence of Porcine Placenta Enzymatic Hydrolysate Index
Component

| | | RT | | | Content in |
|---|---|---|---|---|---|
| No. | m/z | (min) | Charge | Sequence | Hydrolysate (ppm) |
| PEP-1 | 445.27 | 9.4 | 1 | VVVE (SEQ ID No: 1) | 10 to 19 ppm |
| PEP-2 | 355.70 | 14.3 | 2 | DGLHLR (SEQ ID NO: 2) | 4-10 ppm |
| PEP-3 | 465.71 | 15.3 | 2 | DDFNPSVH (SEQ ID No: 3) | 10 to 21 ppm |

After peptides having the same mass and ms/ms ionization forms as the peptides identified from the porcine placenta enzymatic hydrolysate were synthesized from Anygen (www.anygen.com), the experiment was carried out.

It was confirmed that the contents of PEP-1, PEP-2 and PEP-3 peptides in the hydrolysate were 1 ppm to 25 ppm.

### 4.2. Verification of Peptide

Through the verification of PEP-1, PEP-2, and PEP-3 peptides, a peptide verification test was carried out to confirm that the peptides synthesized from Anygen were identical to peptides present in the porcine placenta enzymatic hydrolysate.

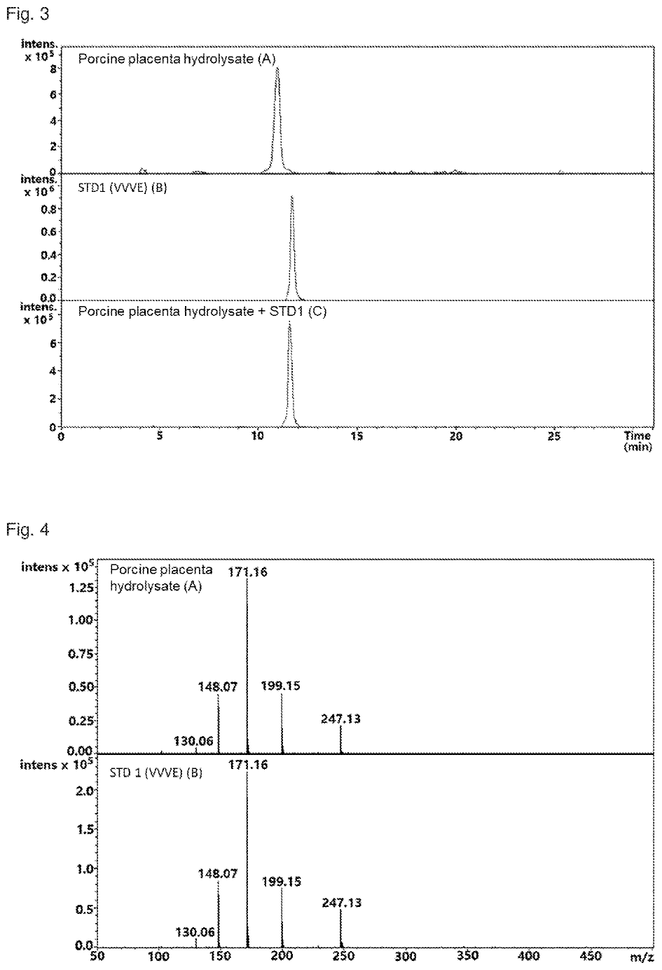

In a case of PEP-1, verification of the peptide was carried out by two methods. As the first method, a chromatogram of the porcine placenta enzymatic hydrolysate and peptide (VVVE) was analyzed. The chromatogram was confirmed by spiking (A) porcine placenta hydrolysate, (B) peptide (VVVE), and (C) peptide synthesized with the porcine placenta hydrolysate. As a result, it was determined that the porcine placenta hydrolysate and the synthetic peptide have the same peak. Thus, it was confirmed that the peptide is consistent with ingredients present in the porcine placenta hydrolysate (FIG. 3). As the second method, MS/MS patterns of the porcine placenta enzymatic hydrolysate and the peptide (VVVE) were confirmed. As a result, the MS/MS pattern of the porcine placenta enzymatic hydrolysate coincides with the MS/MS pattern of the peptide (VVVE), so that it was confirmed that the peptide is consistent with ingredients present in the porcine placenta hydrolysate (FIG. 4).

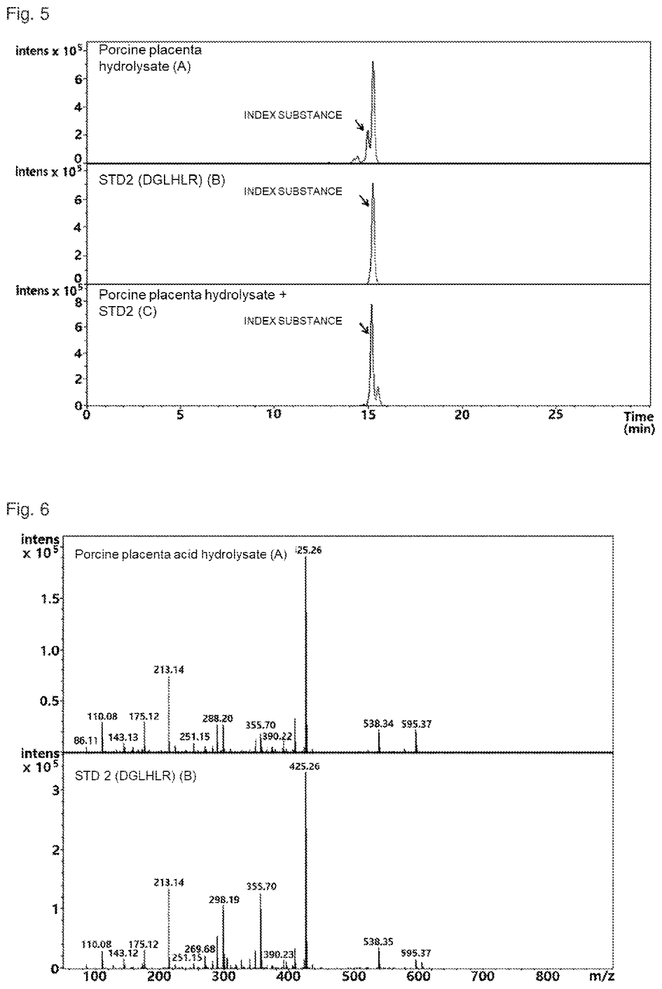

As the first method, in a case of PEP-2, a chromatogram of the porcine placenta enzymatic hydrolysate and peptide (DGLHLR) was analyzed. The chromatogram was confirmed by spiking (A) porcine placenta hydrolysate, (B) peptide (DGLHLR), and (C) peptide synthesized with the porcine placenta hydrolysate. As a result, it was confirmed that the porcine placenta hydrolysate and the synthetic peptide have the same peak. Thus, it was confirmed that the peptide is consistent with ingredients present in the porcine placenta hydrolysate (FIG. 5). In addition, as the second method of the verification of peptide, MS/MS patterns of the porcine placenta enzymatic hydrolysate and the peptide (DGLHLR) were confirmed. As a result, the MS/MS pattern of the porcine placenta enzymatic hydrolysate coincides with the MS/MS pattern of the peptide (DGLHLR), so that it was confirmed that the peptide is consistent with ingredients present in the porcine placenta hydrolysate (FIG. 6).

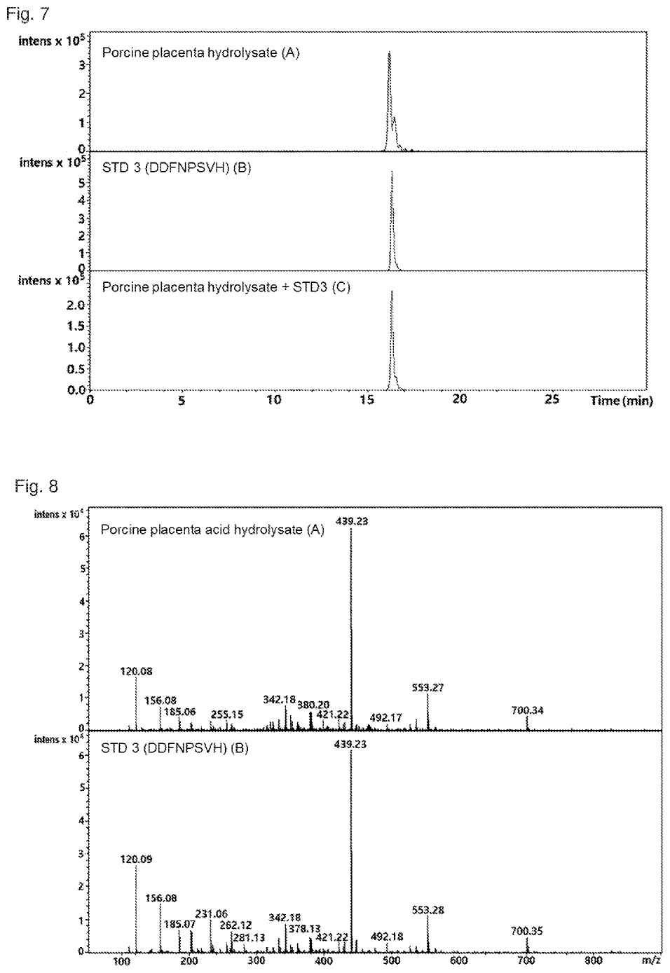

As the first method, in a case of PEP-3, a chromatogram of the porcine placenta enzymatic hydrolysate and peptide (DDFNPSVH) was analyzed. The chromatogram was confirmed by spiking (A) porcine placenta hydrolysate, (B) peptide (DDFNPSVH), and (C) peptide synthesized with the porcine placenta hydrolysate. As a result, it was confirmed that the porcine placenta hydrolysate and the synthetic peptide have the same peak. Thus, it was confirmed that the peptide is consistent with ingredients present in the porcine placenta enzymatic hydrolysate (FIG. 7). In addition, as the second method of the verification of peptide, MS/MS patterns of the porcine placenta enzymatic hydrolysate and the peptide (DDFNPSVH) were confirmed. As a result, the MS/MS pattern of the porcine placenta enzymatic hydrolysate coincides with the MS/MS pattern of the peptide (DDFNPSVH), so that it was confirmed that the peptide is consistent with ingredients present in the porcine placenta hydrolysate (FIG. 8).

### 4.3. HepG2 Cytotoxicity Evaluation of Peptide

After culturing a HepG2 hepatoma cell line, three kinds of synthetic peptides were added by concentration, and MTT (methylthiazol tetrazolium bromide, Sigma Aldrich) was carried out to confirm cell viability. An appropriate amount (1×10⁵/well) of cells was seeded on each well in a 24-well plate (BD, Falcon) to treat a sample according to the concentration, and cell viability was confirmed after cultivation for 24 hours in a 37° C. incubator. After reaction was performed for 4 hours using an MTT solution (3-(4,5-dimethylthiazol-2-yl)-2,5-diphenyl tetrazolium bromide), 400 μl of DMSO was added at each time to dissolve insoluble formazan crystals, and light absorbance was measured using an ELISA reader (TECAN, Infinite M200 pro) at a wavelength of 570 nm.

As a result of confirming cytotoxicity of three kinds of peptides, PEP-1, PEP-2, and PEP-3 all showed non-toxicity up to 10 μg/ml (Table 5).

### 4.4. Hepatocyte Protection Ability of Peptide

HepG2 cells, which are hepatoma cell lines, were dispensed in the 24-well plate at 1×10⁵/well and cultured. Thereafter, in order to confirm hepatocyte protection ability of three kinds of synthetic peptides, the peptides were treated by concentration, cultured for 23 hours, and then 10 mM of t-BHP was treated for damage to the hepatocytes to culture the same for 90 minutes. After an MTT solution (3-(4,5-dimethylthiazol-2-yl)-2,5-diphenyl tetrazolium bromide) was added and reacted for 4 hours, 400 μl of DMSO was added at each time to dissolve insoluble formazan crystals, and light absorbance was measured using an ELISA reader (TECAN, Infinite M200 pro) at a wavelength of 570 nm.

As a result, PEP-2 and PEP-3 showed high hepatocyte protection ability of 26% and 20%, respectively at 10 μμg/ml (Table 5).

### 4.5. Measurement of Liver Function Test Index AST of Peptide

In order to confirm AST, three kinds of synthetic peptides were treated in each well (1×10⁵/well) by concentration and cultured for 23 hours, and then 20 mM of t-BHP was treated to culture the same for 3 hours. Thereafter, a supernatant was taken to measure AST using an Aspartate transaminase (AST or SGOT) Activity Colorimetric Assay Kit (BIOVISION; K753-100). In addition, after the supernatant was removed for quantification of cells and reaction was performed for 4 hours using an MTT solution (3-(4,5-dimethylthiazol-2-yl)-2,5-diphenyl tetrazolium bromide), 400 μl of DMSO was added at each time to dissolve insoluble formazan crystals, and light absorbance was measured using an ELISA reader (TECAN, Infinite M200 pro) at a wavelength of 570 nm.

As a result, each of PEP-2 and PEP-3 showed the most remarkable AST inhibitory ability at 10 μg/ml, and values thereof were 34% and 14% (Table 6). As shown in the results of hepatocyte protection ability, it was confirmed that PEP-2 showed the best efficacy.

### 4.6. Measurement of Liver Function Test Index ALT of Peptide

In order to confirm ALT, three kinds of synthetic peptides were treated in each well (1×10⁵/well) by concentration and cultured for 23 hours, and then 20 mM of t-BHP was treated to culture the same for 3 hours. Thereafter, a supernatant was taken to measure ALT using an Aspartate transaminase (ALT or SGPT) Activity Colorimetric/Fluorometric Kit (BIOVISION; K752-100). In addition, after the supernatant was removed for quantification of cells and reaction was performed for 4 hours using an MTT solution (3-(4,5-dimethylthiazol-2-yl)-2,5-diphenyl tetrazolium bromide), 400 μl of DMSO was added at each time to dissolve insoluble formazan crystals, and light absorbance was measured using an ELISA reader (TECAN, Infinite M200 pro) at a wavelength of 570 nm.

As a result, it was confirmed that PEP-2 has excellent ALT inhibitory ability. The concentration of the sample showing the maximum efficacy was 1 μg/ml, and a value thereof was 34% (Table 5).

TABLE 5
Liver Health Improvement Effect Test of Porcine Placenta-derived Peptide

| | | Sample | | | | |
|---|---|---|---|---|---|---|
| | | Concentration | | PEP-1 | PEP-2 | PEP-3 |
| Synthetic Sample | Sequence | — | | VVVE | DGLHLR | DDFNPSVH |
| Test | Cytotoxicity | 0.01 to 1000 | ug/ml | Equal to or | Equal to or | Equal to or |
| Concentration | (HepG2) | | | less than 10 | less than 10 | less than 10 |
| Setting | | | | ug/ml | ug/ml | ug/ml |
| Liver index | Hepatocyte | 0.1 | ug/ml | ND | 15% | 9% |
| | Protection | 1 | ug/ml | ND | 20% | 20% |
| | Ability | 10 | ug/ml | 7% | 26% | 20% |
| | AST Inhibitory | 0.1 | ug/ml | ND | 4% | ND |
| | Ability | 1 | ug/ml | ND | 15% | 5% |
| | | 10 | ug/ml | 2% | 34% | 14% |
| | ALT Inhibitory | 0.01 | ug/ml | ND | 21% | ND |
| | Ability | 0.1 | ug/ml | ND | 30% | ND |
| | | 1 | ug/ml | ND | 34% | ND |

### Example 5. Evaluation of Liver Improvement In-Vitro Efficacy of Porcine Placenta Enzymatic Hydrolysate and Porcine Placenta Acid Hydrolysate

The present inventors performed a cytotoxicity experiment of the porcine placenta enzymatic hydrolysate and the porcine placenta acid hydrolysate by treating the porcine placenta enzymatic hydrolysate and the porcine placenta acid hydrolysate with human hepatoma cell lines, and then measuring cell viability. Briefly, a HepG2 human hepatoma cell line was cultured in cell culture flask, and when it reached 80% confluence, the HepG2 human hepatoma cell line was dispensed in a 24-well plate by the number of cells of 1.5×10⁵. After cultivation for 48 hours, each test substance for each concentration was treated and further cultured for 24 hours. Thereafter, reaction was performed for 4 hours using an MTT solution (3-(4,5-dimethylthiazol-2-yl)-2,5-diphenyl tetrazolium bromide), 400 μl of DMSO was added at each time to dissolve insoluble formazan crystals, and light absorbance was measured using an ELISA reader (TECAN, Infinite M200 pro) at a wavelength of 570 nm.

As a result, it was confirmed that the porcine placenta enzymatic hydrolysate and the porcine placenta acid hydrolysate were non-toxic up to a concentration of 0.5 mg/ml of the total nitrogen (Table 6).

Further, the present inventors performed an experiment to confirm whether the porcine placenta enzymatic hydrolysate and the porcine placenta acid hydrolysate have a liver protection effect. Briefly, a HepG2 human hepatocyte cell line was cultured in a cell culture flask, and when it reached 80% confluence, the HepG2 human hepatocyte cell line was dispensed in a 24-well plate by the number of cells of 1.5×10⁵. After cultivation for 48 hours, each test substance for each concentration was treated and further cultured for 23 hours. Thereafter, the cells were treated with tert-Butyl hydroperoxide (t-BHP, 10 mM) at the same time together with the sample for 1 hour and 30 minutes. After 1 hour and 30 minutes of the treatment at the same time, MTT was compared with a liver damage treatment group damaged by t-BHP to confirm hepatocyte protection ability.

As a result, it was confirmed that t-BHP was treated with hepatocytes to generate a large amount of ROS and damage hepatocytes. However, when the porcine placenta enzymatic hydrolysate was treated, hepatocytes were protected by about 40% at a concentration of 0.05 mg/ml of the total nitrogen, and the porcine placenta acid hydrolysate was effectively protected by about 21% at a concentration of 0.05 mg/ml of the total nitrogen (Table 6).

Further, the present inventors performed an experiment to confirm whether the porcine placenta enzymatic hydrolysate and the porcine placenta acid hydrolysate have an effect on inhibiting hepatotoxicity. A HepG2 human hepatoma cell line was cultured in a cell culture flask and when it reached 80% confluence, the HepG2 human hepatoma cell line was divided in a 24-well plate by the number of cells of 1.5×10⁵. After cultivation for 48 hours, each test substance for each concentration was treated and further cultured for 23 hours. After 23 hours, cells were treated with t-BHP (20 mM) for 3 hours at the same time, and a supernatant was obtained after 3 hours to confirm an amount of aspartate transfection (AST) and alanine transfection (ALT), which are liver damage indices, using AST and ALT activity kits. In addition, an MTT solution was treated to confirm and correct a number of cells.

As a result, when the porcine placenta enzymatic hydrolysate was treated at a concentration of 0.1 mg/ml of the total nitrogen, it was confirmed that AST was inhibited to 91%, and when the porcine placenta acid hydrolysate was treated at a concentration of 0.1 mg/ml of the total nitrogen, it was confirmed that AST was inhibited to 93%. In addition, when the porcine placenta enzymatic hydrolysate was treated at a concentration of 0.1 mg/ml of the total nitrogen, it was confirmed that ALT was inhibited by 23%, whereas when the porcine placenta acid hydrolysate was treated, it was confirmed that no change was made (Table 6).

TABLE 7
Analysis of Liver Function Improvement Efficacy of Porcine Placenta
Enzymatic Hydrolysate And Porcine Placenta Acid Hydrolysate

| | | Hepatocyte Protection | AST Inhibitory | ALT Inhibitory |
|---|---|---|---|---|
| Classification | Cytotoxicity | Ability | Ability | Ability |
| Enzymatic Hydrolysate | Non- | Approximately 40% | 91% | 23% |
| | toxicity | Protection | (0.1 mg/ml) | (0.1 mg/ml) |
| | (0.5 mg/ml) | (0.05 mg/ml) | | |
| Porcine Placenta Acid | Non- | Approximately 21% | 93% | No Change |
| Hydrolysate | toxicity | Protection | (0.1 mg/ml) | (0.1 mg/ml) |
| | (0.5 mg/ml) | (0.05 mg/ml) | | |

### Example 6. Analysis of Properties of Mixture of Porcine Placenta Enzymatic Hydrolysate and Porcine Placenta Acid Hydrolysate

In order to confirm properties of a mixture in which the porcine placenta enzymatic hydrolysate and the porcine placenta acid hydrolysate are mixed at different ratios, the present inventors performed an experiment of mixing the porcine placenta enzymatic hydrolysate and the porcine placenta acid hydrolysate at a weight ratio of 1:1, 1:2, 1:3, and 1:4, and analyzing the nitrogen content and the amino acid content thereof.

As a result, it was confirmed that as a mixing ratio of the porcine placenta acid hydrolysate increases, the content of the amino acid increases (Table 7). Thereafter, in the experiment, a weight ratio of 1:3 in the mixture of the porcine placenta enzymatic hydrolysate and the porcine placenta acid hydrolysate was selected, and an efficacy evaluation was performed on the mixture at various concentrations of low, medium, and high concentrations.

TABLE 7
Analysis of Porcine Placenta Enzymatic Hydrolysate
And Porcine Placenta Acid Hydrolysate

| | Total | Amino | Amino | |
|---|---|---|---|---|
| | Nitrogen | Acid | Acid | Peptide |
| Classification | (mg/mL) | (mg/mL) | % | % |
| Enzyme + Acid (1:1) | 5.47 | 15.73 | 59.4 | 40.6 |
| Enzyme + Acid (1:2) | 5.52 | 17.54 | 65.8 | 34.2 |
| Enzyme + Acid (1:3) | 5.58 | 18.10 | 66.4 | 33.6 |
| Enzyme + Acid (1:4) | 5.61 | 18.62 | 68.2 | 31.8 |

$$Amino acid ( \% ) = \frac{\begin{matrix}( Molecular weights of nitrogen and amino acid / \\ Molecular weight of amino acid ) \times \\ Content of amino acid\end{matrix}}{Dilution factor} \times 100 \qquad [ Equation ]$$

$$Peptide ( \% ) = 100 \% - Amino acid \%$$

### Example 7. Evaluation of Alcoholic Liver Damage In-Vivo Efficacy of Porcine Placenta Enzymatic Hydrolysate, Porcine Placenta Acid Hydrolysate, and Mixture of Porcine Placenta Enzymatic Hydrolysate and Porcine Placenta Acid Hydrolysate

### 7.1. Sample Preparation

A powder sample was prepared by spray-drying the porcine placenta enzymatic hydrolysate, the porcine placenta acid hydrolysate, and the mixture of the porcine placenta enzymatic hydrolysate and the porcine placenta acid hydrolysate Accordingly, in order to confirm properties of the powder sample, a nitrogen content and an amino acid content of the powder sample were analyzed (Table 8).

TABLE 8
Porcine Placenta Enzymatic Hydrolysate, Porcine Placenta
Acid Hydrolysate, And Mixture of Porcine Placenta Enzymatic
Hydrolysate And Porcine Placenta Acid Hydrolysate

| | Total | Amino | Amino | |
|---|---|---|---|---|
| | Nitrogen | Acid | Acid | Peptide |
| Classification | (mg/mL) | (mg/mL) | % | % |
| Enzymatic Hydrolysate | 46.79 | 125.25 | 42.5 | 57.5 |
| Porcine Placenta Acid | 35.70 | 197.38 | 81.2 | 18.8 |
| Hydrolysate | | | | |
| Mixture of Enzyme and Acid | 39.27 | 176.27 | 64.0 | 36.0 |
| Hydrolysate (1:3) | | | | |

$$Amino acid ( \% ) = \frac{\begin{matrix}( Molecular weights of nitrogen and amino acid / \\ Molecular weight of amino acid ) \times \\ Content of amino acid\end{matrix}}{Dilution factor} \times 100 \qquad [ Equation ]$$

$$Peptide ( \% ) = 100 \% - Amino acid \%$$

### 7.2. Animal Breeding

In the present invention, 7-8-week-old SD type white rats (about 250 gm) were purchased, followed by observing body weight change and general health status of the rats through a one-week quarantine and purification period, thereby using a health individual of the rats. During the experiment period, all groups except the first group were given a free intake of alcohol diet (Lieber DeCalie Liquid Ethanol Diet) for rats, which was radiation-sterilized, for 4 weeks, and alcohol was further administered orally twice a week (tuesday/thursday) in 4th weeks, and no drinking water was supplied separately. A temperature of a breeding room was maintained at 23±2° C., relative humidity was maintained at 40 to 60%, and the number of ventilation was made 10 to 12 times per hour. In addition, light was adjusted such that a light period and a dark period were 12 hours.

### 7.3. Preparation Method of Alcohol Diet (Lieber DeCalie Liquid Ethanol Diet)

A preparation method of alcohol diet is as follows: 1) Add 821 ml of water to a necessary weight of powder feed (132.28 g) and 67 ml of alcohol (spirit) in a beaker; 2) add sufficient water and sufficiently stir the water so that there are no lumps; 3) add water up to 1 L marked on the beaker; 4) sufficiently stir the feed, add the feed to the blender, and mix the feed for 30 seconds; and 5) use a feeding tube (120 ml).

When a regular feeder is used for liquid diet, the loss may be large because the liquid diet overflows or easily sticks to the animal's body. In addition, since an area in contact with air is increased, highly volatile alcohol may be blown away or the diet thereof may be oxidized, thereby greatly affecting the experiment. Therefore, in this experiment, a feeder for liquid diet was used.

### 7.4. Test Group

Test groups are as follows.
(1) Normal Control Group
(2) Negative Control Group: Additionally administer alcohol diet (free meals for 4 weeks)+30% alcohol (1.4 g/kg, PO) twice in the 4th week
(3) Positive Control Group Additionally administer silymarin (100 mg/kg/day, PO) (Yang et al, 2015)+30% alcohol (1.4 g/kg, PO) twice in the 4th week
(4) High-Dose Group of Mixture of Porcine Placenta Enzymatic Hydrolysate And Porcine Placenta Acid Hydrolysate (1:3): Additionally administer 2952 mg/kg/day, PO+alcohol diet (free meals for 4 weeks)+30% alcohol (1.4 g/kg, PO) twice in the 4th week
(5) Medium-Dose Group of Mixture of Porcine Placenta Enzymatic Hydrolysate And Porcine Placenta Acid Hydrolysate: Additionally administer 1771 mg/kg/day, PO+alcohol diet (free meals for 4 weeks)+30% alcohol (1.4 g/kg, PO) twice in the 4th week
(6) Low-Dose Group of Mixture of Porcine Placenta Enzymatic Hydrolysate And Porcine Placenta Acid Hydrolysate (1:3): Additionally administer 590 mg/kg/day, PO+alcohol diet (free meals for 4 weeks)+30% alcohol (1.4 g/kg, PO) twice in the 4th week
(7) High-Dose Group of Porcine Placenta Enzymatic Hydrolysate: Additionally administer 2511 mg/kg/day, PO+alcohol diet (free meals for 4 weeks)+30% alcohol (1.4 g/kg, PO) twice in the 4th week
(8) High-Dose Group of Porcine Placenta Acid Hydrolysate: Additionally administer 3282 mg/kg/day, PO+alcohol diet (free meals for 4 weeks)+30% alcohol (1.4 g/kg, PO) twice in the 4th week
   7.5. Efficacy of Improving Liver Function from Alcoholic Liver Damage

The present inventors performed an experiment for 4 weeks according to a protocol after preliminary breeding test animals for about 1 week. Test samples were prepared at a daily dose of a predetermined concentration and administered orally once, and the alcohol diet was prepared according to a predetermined preparation method and supplied daily to feed the same freely. In the 4th week, the test samples were additionally administered orally twice at a predetermined dose. After 4 weeks of the test, the blood was depleted by cardiac puncture after fasting for 12 hours, and the blood was centrifuged at 3000 rpm, and then quantified with an alcohol kit and an acetaldehyde kit using serum to compare and analyze the blood concentration. In addition, the blood, which was collected for hepatotoxicity evaluation by cardiac puncture, was put in a heparin tube and centrifuged at 10,000 rpm for 10 minutes, and then a liver enzyme level was measured to evaluate hepatotoxicity. A part of the tissue was taken to evaluate liver tissue changes and intrahepatic ADH and ALDH enzyme activity. Through all results, significance between the test groups was verified with Student's t-test and ANOVA test based on average and standard errors.

In the present invention, an attempt was made to verify the efficacy of porcine placenta on the overall liver condition of liver synthesis ability as well as liver inflammation by measuring an amount of albumin and total protein that represent synthesis ability of liver, including ALP, ALT, and AST representing liver disease in a serum index. The measurement values for each serum index are shown in Table 9.

As a result, serum albumin is an index indicating liver synthesis ability together with the total protein amount, and there was no significant difference in the index for each group.

Alkaline phosphatase (ALP) is mainly distributed in the liver, bone tissue, intestinal tract, white blood cells, and the like, and the increase in levels is mainly due to the liver and bone tissue. In the present invention, when test substances (silymarin, porcine placenta extract) were administered, the ALP was tended to be decreased compared to the negative control group, but in particular, it was confirmed that the ALP was significantly decreased in the medium-dose group of the mixture of the porcine placenta enzymatic hydrolysate and the porcine placenta acid hydrolysate; and the porcine placenta enzymatic hydrolysate.

ALT and AST are representative amino acid transferases that exhibit liver function. It was confirmed that the ALT level in the alcohol-administered negative control group was increased by about 3.5 times compared to the normal control group, and when the test substances (silymarin, porcine placenta extract) were administered, the ALT level was decreased compared to the negative control group. In particular, when the mixture of the porcine placenta enzymatic hydrolysate and the porcine placenta acid hydrolysate was administered, the ALT level was dose-dependently decreased, and the ALT level was most remarkably decreased in the high dose of the mixture of the porcine placenta enzymatic hydrolysate and the porcine placenta acid hydrolysate.

In addition, in the alcohol-administered negative control group, the AST level was significantly increased due to hepatotoxicity compared to the normal control group, and the AST level was significantly decreased in all test groups except for the high dose of enzymatic hydrolysate in the test substances. In particular, when the mixture of the porcine placenta enzymatic hydrolysate and the porcine placenta acid hydrolysate was administered, it was confirmed that the AST level was dose-dependently decreased as in ALT.

TABLE 9
Serum Biochemical Test of Porcine Placenta Extract for Alcoholic Liver Damage

| | | | | ALT | AST | Total |
|---|---|---|---|---|---|---|
| | | Albumin | ALP | (GPT) | (GOT) | Protein |
| Normal Group | | 2.24 ± | 483.60 ± | 33.66 ± | 81.37 ± | 5.32 ± |
| | | 0.13 | 119.02 | 3.11 | 3.24 | 0.31 |
| Negative Control Group | | 2.38 ± | 633.73 ± | 115.56 ± | 235.03 ± | 5.44 ± |
| | | 0.08 | 91.97 | 38.96 | 113.98 | 0.14 |
| Positive Control Group | | 2.37 ± | 555.63 ± | 83.94 ± | 126.70 ± | 5.24 ± |
| | | 0.13 | 108.75 | 28.54 | 23.81 | 0.28 |
| Mixture of Porcine Placenta | High | 2.33 ± | 552.89 ± | 73.64 ± | 139.17 ± | 5.19 ± |
| Enzyme And Acid (1:3) | Dose | 0.22 | 94.56 | 10.15 | 17.64 | 0.46 |
| | Middle | 2.34 ± | 490.23 ± | 68.76 ± | 123.97 ± | 5.22 ± |
| | Dose | 0.18 | 82.20 | 21.50 | 25.76 | 0.34 |
| | Low | 2.39 ± | 568.11 ± | 56.91 ± | 122.83 ± | 5.32 ± |
| | Dose | 0.12 | 85.03 | 13.99 | 17.23 | 0.27 |

| Enzymatic Hydrolysate | 2.42 ± | 471.36 ± | 82.54 ± | 227.31 ± | 5.42 ± |
|---|---|---|---|---|---|
| | 0.19 | 99.17 | 31.70 | 90.21 | 0.35 |
| Porcine Placenta Acid Hydrolysate | 2.42 ± | 556.39 ± | 78.81 ± | 169.36 ± | 5.31 ± |
| | 0.10 | 55.71 | 11.32 | 10.36 | 0.31 |

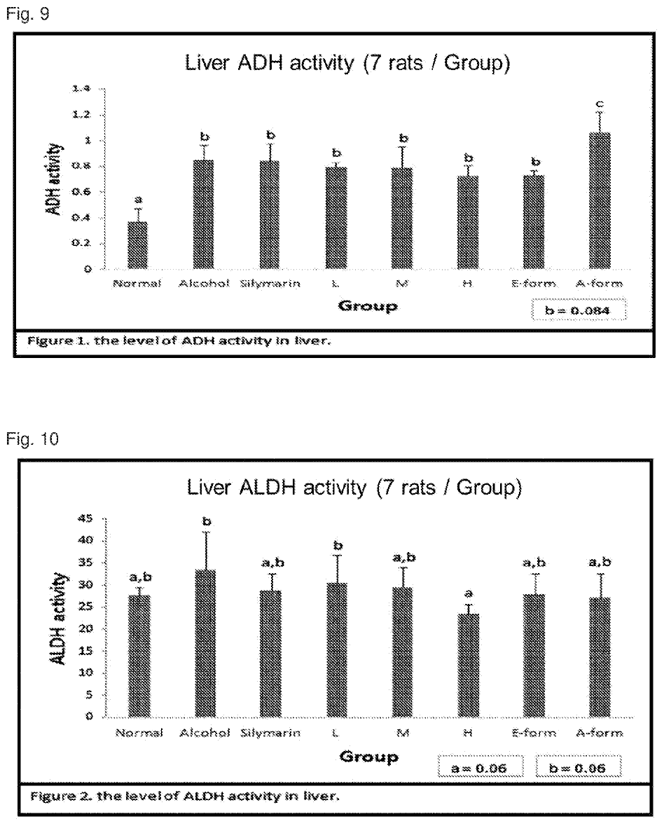

In addition, as a result of measuring activity of ADH, which is an alcohol metabolizing enzyme in liver tissue, the ADH activity was increased in the negative control group (0.85) to which alcohol was administered compared to the normal control group (0.37) (FIG. 9). In addition, the ADH activity was tended to be decreased in the order of silymarin>low dose of the porcine placenta mixture>medium dose of the porcine placenta mixture>high dose of the porcine placenta mixture. As a result, the ADH activity in the mixture was further decreased than in an independent treatment group of the porcine placenta enzymatic hydrolysate and the porcine placenta acid hydrolysate. However, in the group to which the porcine placenta acid hydrolysate was administered, the enzyme activity was significantly increased (FIG. 9).

In addition, as a result of measuring activity of ALDH, which is an alcohol metabolizing enzyme in liver tissue, the ALDH activity was increased in the negative control group to which alcohol was administered compared to the normal control group, and the ALDH activity increased by the enzyme-inducing action was generally decreased in the group to which silymarin or porcine placenta extract was administered (FIG. 10). In the mixture of the porcine placenta enzymatic hydrolysate and the porcine placenta acid hydrolysate, the enzyme-inducing action of alcohol was dose-dependently inhibited, and the ALDH activity was most clearly inhibited in the high-dose group of the porcine placenta mixture (FIG. 10). The effects of the porcine placenta enzymatic hydrolysate and the porcine placenta acid hydrolysate were almost similar (FIG. 10). In the case of the mixture, the ALDH activity of ALDH was more inhibited than that in the independent treatment group.

### Preparation Example 1 Preparation of Health Functional Food Composition

After adding water to liquid fructose (0.5 wt %), oligosaccharide (2 wt %), sugar (2 wt %), and salt (0.5 wt %) to make balance, the resultant was uniformly mixed with a solution, which is obtained by dissolving a mixture of a porcine placenta enzymatic hydrolysate and a porcine placenta acid hydrolysate (mixed at a weight ratio of 1:3) in distilled water at a concentration of 100 mg/100 ml (0.1 wt %) or 15,000 mg/100 mL (15 wt %), and was instantaneously sterilized to prepare a health functional beverage.

### Preparation Example 2 Preparation of Pharmaceutical Composition

1 mg of a mixture of a porcine placenta enzymatic hydrolysate and a porcine placenta acid hydrolysate (mixed in a weight ratio of 1:3) was dissolved in 5 ml of distilled water or physiological saline, sterilized, and prepared as an injection. Alternatively, the mixture was prepared as a powder formulation after lyophilization in a vial. A capsule was prepared by filling a gelatin capsule with 100 mg of the porcine placenta hydrolysate, 100 mg of corn starch, 100 mg of lactose, and 2 mg of magnesium stearate.

1. A health functional food composition for liver protection comprising a mixture of a porcine placenta enzymatic hydrolysate and a porcine placenta acid hydrolysate as an effective ingredient.

2. A health functional food composition for preventing or alleviating liver damage caused by alcohol, drug addiction, or hangover, the composition comprising a mixture of a porcine placenta enzyme hydrolysate and a porcine placenta acid hydrolysate as an effective ingredient.

3. The composition of claim 1, wherein the porcine placenta enzymatic hydrolysate comprises one or more peptides having an amino acid sequence selected from the group consisting of SEQ ID Nos: 1 to 3.

4. The composition of claim 1, wherein the porcine placenta enzymatic hydrolysate is prepared by treating proteinase.

5. The composition of claim 1, wherein the porcine placenta acid hydrolysate is prepared by treating acid.

6. The composition of claim 5, wherein the acid is hydrochloric acid, sulfuric acid, acetic acid, or citric acid.

7. The composition of claim 1, wherein the porcine placenta enzymatic hydrolysate and the porcine placenta acid hydrolysate are mixed at a weight ratio of 1:0.1 to 10.


SEQUENCE LISTING (3 sequences)

SEQ ID NO: 1 - protein, 4 aa, Artificial Sequence
  PEP-1 (Peptide-1)
VVVE

SEQ ID NO: 2 - protein, 6 aa, Artificial Sequence
  PEP-2 (Peptide-2)
DGLHLR

SEQ ID NO: 3 - protein, 8 aa, Artificial Sequence
  PEP-3 (Peptide-3)
DDFNPSVH